(19) United States
(12) Patent Application Publication
(10) Pub. No.: US 2026/0042611 A1
(43) Pub. Date: Feb. 12, 2026

(54) ELIMINATING MICROBIAL HYDROGEN CONSUMPTION IN SUBSURFACE HYDROGEN STORAGE

(71) Applicant: EXXONMOBIL TECHNOLOGY AND ENGINEERING COMPANY, Spring, TX (US)

(72) Inventor: Alex G. LEE, New Hope, PA (US)

(21) Appl. No.: 19/296,337

(22) Filed: Aug. 11, 2025

Related U.S. Application Data
(60) Provisional application No. 63/682,202, filed on Aug. 12, 2024.

(51) Int. Cl. B65G 5/00; A61L 2/20

(52) U.S. Cl. CPC B65G 5/00; A61L 2/20

(57) ABSTRACT

Hydrogen consuming microorganisms can cause operational challenges for subterranean hydrogen storage. The systems and methods described herein provide sterilization techniques that include acidifying a subterranean reservoir prior to injection of hydrogen. Acidification may be facilitated by, at least partially, saturating a fluid with an acidifying agent such as carbon dioxide. The subterranean reservoir's fluid may be reduced to 3 pH to 4 pH. Hydrogen may then be injected into the subterranean reservoir for storage.

### CROSS-REFERENCE TO RELATED APPLICATIONS

This Non-Provisional Patent application claims priority to U.S. Provisional Application 63/682,202, filed Aug. 12, 2024, and titled “Eliminating Microbial Hydrogen Consumption in Subsurface Hydrogen Storage,” the entire contents of which is hereby incorporated by reference into the present application.

### FIELD OF THE INVENTION

The present disclosure provides methods and processes for subsurface storage of Hydrogen.

### BACKGROUND

Underground hydrogen storage is a storage technology for the large-scale and seasonal storage of hydrogen. However, hydrogen consuming microorganisms including methanogens, sulfate reducers, and acetogens can cause operational challenges for hydrogen recovery, gas injectivity, and corrosion. Active research efforts are underway in industry, academia, and national labs to develop hydrogen storage site selection criteria, i.e., site depth, temperature, brine salinity, etc. that would reduce the risk of microbial activities. For example, Gregory et al. in “Subsurface Microbial Hydrogen Cycling: Natural Occurrence and Implications for Industry” which published in the February 2019 issue of Microorganisms, suggests that both biotic hydrogen production (e.g., via fermentation, nitrogen fixation, anaerobic carbon monoxide oxidation, or phosphite oxidation) and abiotic hydrogen production (e.g., naturally occurring graphitization, radiolysis, serpentinization, catalases, and so forth) can sustain microbial communities.

Similarly, Thaysen et al., wrote in “Estimating microbial growth and hydrogen consumption in hydrogen storage in porous media,” which published in issue 151 of Renewable and Sustainable Energy Reviews that while subsurface storage of hydrogen may provide a viable means to address supply and demand imbalances in the renewable energy sector, hydrogen functions as an electron donor for microbial processes. Thaysen goes on to state that 518 strains of the three major groups of H₂-oxidizing microorganisms had growth conditions that aligned with the in natura environmental conditions (e.g., physical and chemical) of many depleted oil and gas fields (DOGF) and aquifers, which may otherwise be ideal locations for the large-scale and seasonal storage of hydrogen. Notably, Thaysen et al. concludes that salinity is the “most crucial environmental factors” and pH does not constrain the growth of homoacetogens, methanogens, and sulphur species reducing microorganisms (SSRM). Accordingly, microbial growth presents a continuing problem for the viability of underground hydrogen storage.

### BRIEF SUMMARY

The systems and methods described herein relate to a sterilization of subterranean reservoirs to prevent proliferation of at least some hydrogen consuming microorganisms. The sterilization technology is generally characterized by the injection of CO₂, an acid gas mixture, or brine pre-saturated with CO₂ to reduce the reservoir pH to 3 to 4 prior to the introduction of hydrogen.

In one aspect, a method for fluid storage in a subterranean reservoir is described. The method includes extracting reservoir liquid from subterranean reservoir, at least partially saturating the reservoir liquid with CO₂, depositing the at least partially saturated reservoir liquid in the subterranean reservoir at a first depth, where the first depth is positioned below and proximate a primary seal of the subterranean reservoir or caprock of the subterranean reservoir, and injecting a storage fluid including Hydrogen (H₂) into the subterranean reservoir at a second depth, where the second depth is positioned at or below the first depth along the vertical axis of the subterranean reservoir. The method for fluid storage may also include where the reservoir liquid is equilibrated with CO₂ at reservoir pressure which typically ranges between 5 to 100 MPa. The method for fluid storage may also include where at least partially saturating the reservoir liquid with CO₂ includes reducing pH of the reservoir liquid from an initial pH to a pH in a range of 3-4 pH.

In another aspect, a method for fluid storage includes injecting CO₂ into a reservoir liquid of a subterranean reservoir at a reservoir injection depth, where the injecting at least reduces the pH of the reservoir liquid from a native pH to a pH in a range of 3-4, and injecting a storage fluid including Hydrogen (H₂) into the subterranean reservoir including reservoir liquid at a pH of 3-4, where the injecting is at a storage fluid injection depth positioned at or below the reservoir injection depth along a vertical axis of the subterranean reservoir.

In yet another aspect, a method for fluid storage includes extracting surface liquid from a surface accessible body of water, at least partially saturating surface liquid with CO₂, depositing the at least partially saturated surface liquid in the subterranean reservoir at a first depth, where the first depth is positioned below and proximate a primary seal of the subterranean reservoir or caprock of the subterranean reservoir, and injecting a storage fluid including Hydrogen (H₂) into the subterranean reservoir at a second depth, where the second depth is positioned at or below the first depth along the vertical axis of the subterranean reservoir.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 depicts experimental data related to the microbial strain count of homoacetogens, methanogens, and Sulphur species reducing microorganisms (SSRM);

FIG. 2 depicts experimental data related to the pH of aqueous CO₂ at various pressures and temperatures;

FIG. 3 depicts an example process for CO₂-saturated brine injection based subterranean reservoir sterilization, in accordance with aspects described herein;

FIG. 4 illustrates a method 400 in accordance with one embodiment.

FIG. 5 depicts an example process for CO₂ injection based subterranean reservoir sterilization, in accordance with aspects described herein; and

FIG. 6 depicts a method for CO₂ injection based subterranean reservoir sterilization, in accordance with aspects described herein.

### DETAILED DESCRIPTION

Hydrogen has several advantageous properties as a fuel source. For example, combustion of hydrogen has low or no emissions. It is comparatively abundant and has the highest energy density of any commonly used fuel. Long-term hydrogen storage is challenging for multiple reasons. For example, containing diatomic hydrogen is challenging given the molecular size of diatomic hydrogen. Underground hydrogen storage provides a number of advantages over aboveground storage. For example, depleted oil and gas fields (DOGF) provide vast storage capacity with natural resistance to gas leakage. However, underground storage areas (e.g., DOGF, saline formation, or aquifer) can support hydrogen-consuming microbes, which may feed on any hydrogen stored in the storage area. Without effective sterilization, or other microbe inhibition technique, many underground storage sites would have limited viability due to high risk of microbial activities.

Several important hydrogen consuming microbes in subsurface gas storage sites are hydrogenotrophic sulfate reducers (couple H₂-oxidation to sulfate reduction to produce H2S), hydrogenotrophic methanogens (reduce CO₂ to methane), or homoacetogens (couple H₂-oxidation to CO₂ reduction to produce acetate). Microbial growth and H₂ consumption rates vary with nutrient availability and environmental variables such as temperature, salinity, pH, and pressure. Assuming abundant nutrient supply, each strain is adapted to an optimum set of environmental conditions where potentially the greatest growth rates occur. Beyond those conditions, organisms may grow but at reduced rate or they become dormant.

Base unit, unit prefix, and chemical abbreviations are used herein. Unless explicitly stated otherwise, the base unit and unit prefix abbreviations are used consistent with the definitions of the Système International (SI), also referred to as the International System of Units. For example, ‘m’ refers to meter(s) and ‘km’ refers to kilometer(s). Unless explicitly stated otherwise, chemical abbreviations are used consistent with the periodic table of the elements.

Additionally, the term vol % is used herein. This term means percent by volume.

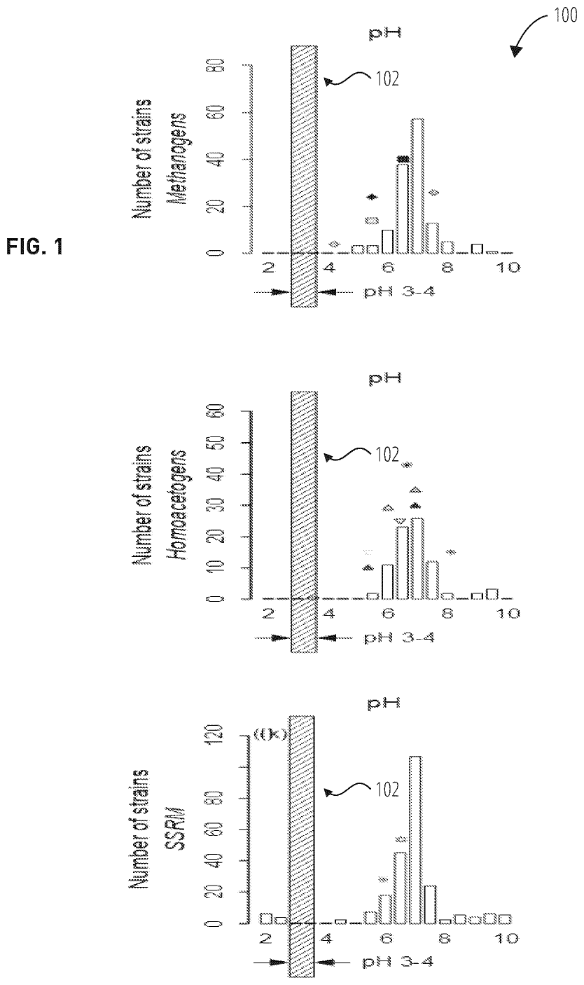

Turning to FIG. 1, graphs depicting the number of hydrogen consuming microbial strain under various pH conditions along with the reservoir conditions of 42 DOGFs are provided. The graphs depicted in FIG. 1 are adapted from data included in ‘Estimating Microbial Growth and Hydrogen Consumption in Hydrogen Storage in Porous Media,’ by Thaysen, McMahon, Strobel, Butler, Heinemann, Ngwenya, Wilkinson, Hassanpouryouzband, McDermott, and Edlmann, which was published in 2021 in Renewable and Sustainable Energy Reviews, vol. 151, having a digital object identifier of 111481 (available at https://doi.org/10.1016/j.rser.2021.111481, which was last visited on Jun. 4, 2024).

In general, FIG. 1 depicts the microbial strain count (i.e., the number of identifiable microbial strains) for methanogens, homoacetogens, and sulphur species reducing microorganisms (SSRM) in a variety of pH conditions. Specifically, FIG. 1 depicts the distribution of optimum growth temperature, critical growth temperature, optimum pH values and critical salinity for 101-143 methanogens strains, 19-88 homoacetogens strains, and 165-277 SSRM strains. As indicated by the addition of bar 102 to the data provided by Thaysen, et al., microbial growth of methanogens, homoacetogens, and sulphur species reducing microorganisms (SSRM) is hindered or prevented in a pH range of 3-4.

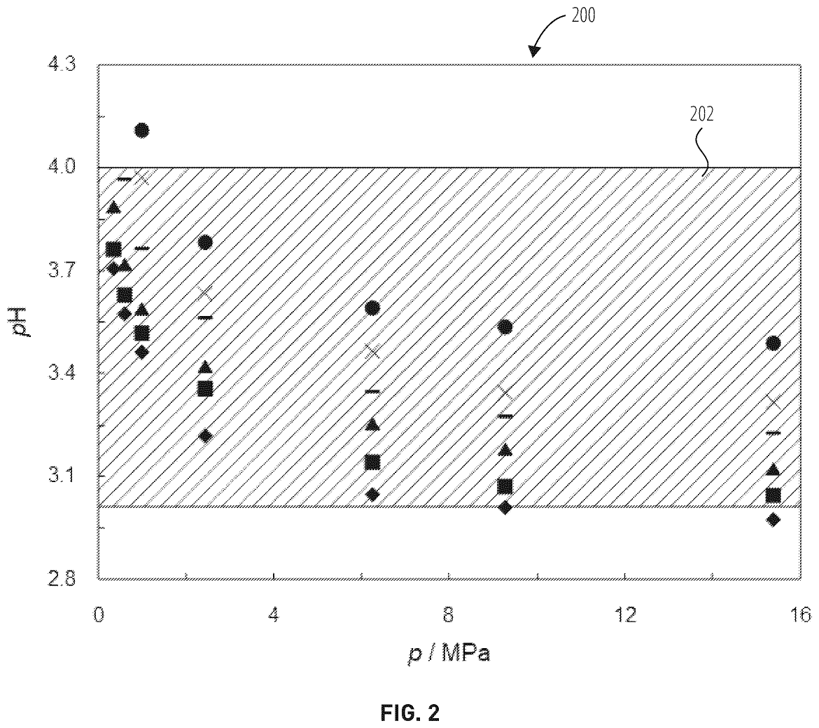

Turning to FIG. 2, a graph depicting the pH of CO₂-saturated H₂O at temperatures between 308 Kelvin (K) and 423K. In the graph each symbol represents a different temperature within the range. Specifically, ♦=308.3K; ▪=323.0K; ▴=343.0K; -=368.1K; X=398.3K; and, •=423.2K. The graph is derived from data in “The pH of CO₂-saturated water at temperatures between 308K and 423K at pressures up to 15 MPa” by Cheng Peng, John P. Crawshaw, Geoffrey C. Maitland, J. P. Martin Trusler, and David Vega-Maza, which was published in The Journal of Supercritical Fluids, Volume 82, 2013, 129-137, ISSN 0896-8446 (available Pages at https://doi.org/10.1016/j.supflu.2013.07.001, which was last visited on Jun. 4, 2024). As emphasized by the addition of bar 202 to the data provided by Peng, et al., pH of 2024EM086-US-saturated H₂O reaches equilibrium of in an approximate range of 3.0 pH and 4.0 pH from 2 MPa and 15 MPa while the fluid temperature remains between 308K and 423K.

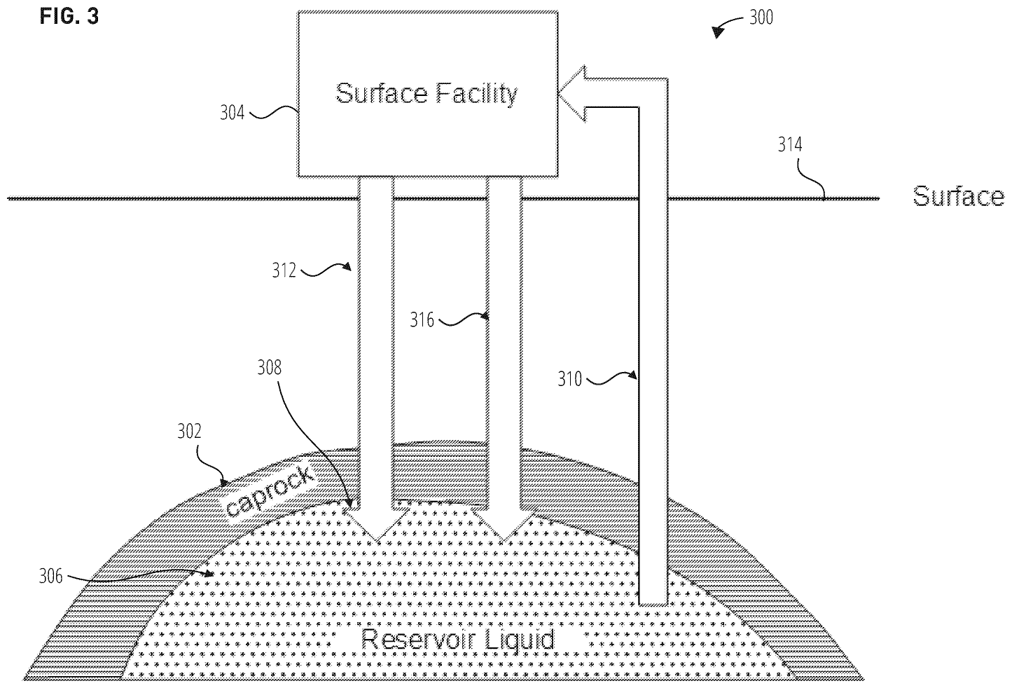

Turning to FIG. 3, a process diagram 300 is depicted, in accordance with aspects describe herein. In general, process diagram 300 includes the saturation of brine with an acidifying agent such as CO₂. In some embodiments, the brine comprises the liquid present in the subterranean reservoir, which is referred to herein as ‘reservoir liquid’ (e.g., reservoir liquid 310). In such an embodiment, the reservoir liquid 310 may be pumped to a surface facility 304 using any suitable means. For example, a rotary drilling rig may be used to penetrate the earth until the borehole reaches the reservoir depth. The borehole may be stabilized by inserting steel casing and securing it with cement or by any other suitable means. For example, an alternative to casing is open-hole completion, where the reservoir section remains uncased, depending on geological conditions and extraction goals. Following this, piping and a pump may be used to transport the reservoir liquid 310 to the surface facility 304.

Additionally, or alternatively, in some embodiments the brine may comprise surface water. For example, fresh water, brackish water, or salt water may be pumped from a suitable surface body of water to the surface facility 304. The salinity of the surface water may be adjusted as necessary, or otherwise treated, based on the subterranean reservoir 306 conditions to create produced formation brine.

Process diagram 300 also includes at least partial saturation of the brine with CO₂. Any suitable means may be used to saturate the brine with CO₂. For example, the brine may be directed into a high-pressure saturator or mixing chamber. The CO₂ gas may be supplied from a high-pressure CO₂ source and can be injected into the brine stream using a gas compressor to ensure that the CO₂ is at a pressure at least equivalent with the subterranean reservoir conditions. In some embodiments, the saturation system is equipped with high-pressure diffusers that introduce the CO₂ into the brine as fine bubbles. The diffusion of CO₂ with fine bubbles may increase the surface area for dissolution of the CO₂. The saturation of brine with CO₂ lowers the pH of the brine from a native pH to between 3.0 pH and 4.0 pH. The acidification of the brine by CO₂-saturation includes multiple chemical processes that form carbonic acid (H₂CO₃) and dissociation of H₂CO₃ into bicarbonate (HCO₃⁻) and hydrogen ions (H⁺). For example, when gaseous CO₂ is introduced into the brine, it dissolves according to Henry's law. The dissolution can be expressed as:

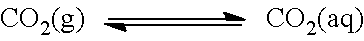

Once dissolved, CO₂ reacts with water to form carbonic acid (H₂CO₃). The general equilibrium can be expressed as:

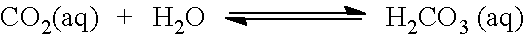

In an aqueous environment H₂CO₃, at least partially, dissociates into bicarbonate (HCO₃⁻), hydrogen ions (H⁺), and carbonate (CO₃²⁻) contributing to the acidification of the water. The dissociation occurs in two steps:

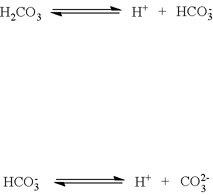

As discussed in relation to FIG. 2, at subterranean reservoir pressures and temperatures the CO₂ saturated water will equilibrate at a pH in the range of 3 to 4.

Process diagram 300 also includes injecting the CO₂-saturated brine 312 into the subterranean reservoir 306. The injection can be completed by any suitable means. For example, the pressurized CO₂-saturated brine may be transported through a network of high-pressure pipelines from the surface facility to the injection site (e.g., the borehole). The pressurized CO₂-saturated brine may be pumped down a high-pressure pipeline to a preset depth. The preset depth may vary based on the position of the subterranean reservoir and the conditions of the subterranean reservoir. For example, CO₂ saturated reservoir liquid or produced formation brine is heavier than in natura reservoir liquid so the CO₂ saturated reservoir liquid or CO₂-saturated produced formation brine can be injected at the top of the subterranean reservoir along a vertical axis. The top of the subterranean reservoir is proximate a lower surface of the caprock. Deposition at the top of the subterranean reservoir contributes to the enhanced mixing of the CO₂ saturated reservoir liquid with in natura reservoir liquid throughout the subterranean reservoir. In some embodiments, the brine is equilibrated with CO₂ at a pressure greater than or equal to the hydrostatic pressure of the storage reservoir. For example, the pressure of the storage reservoir may be 5 MPa or higher. In some embodiments, the pressure of the storage reservoir may be 8 MPa or higher. In some embodiments, the pressure of the storage reservoir may be 12 MPa or higher, such as up to 100 MPa.

Process diagram 300 also includes injecting a storage fluid 316 into the subterranean reservoir 306 for, at least temporary, storage. The storage fluid 316 comprises H₂ in some embodiments. For example, in at least one embodiment, the storage fluid comprises at least 40 vol % H₂. In some embodiments, the storage fluid comprises at least 50 vol % H₂.

The injection of the storage fluid may be facilitated by any suitable means. For example, the H₂ may be pressurized to 350-700 bar at the surface facility. The storage fluid may then be pumped down a high-pressure pipeline to a storage fluid preset depth. The preset depth may vary based on the position of the subterranean reservoir and the conditions of the subterranean reservoir. In some embodiments, the preset depth for the storage fluid is equal to or below the injection depth of the CO₂ saturated reservoir liquid. In some embodiments, the preset depth for the storage fluid injection is at least 1000 m. In some aspects, the preset depth for the storage fluid injection may be in an inclusive range of 1000 m to 7500 m. For example, the preset depth may be 4500 m in an aspect.

In some aspects, the preset depth for the storage fluid injection is proximate the lower surface of caprock 308. Deposition at the top of the subterranean reservoir contributes to the localization of the H₂. Additionally, deposition at the top of the subterranean reservoir reduces viscous fingering throughout the subterranean reservoir.

Although the extraction of reservoir liquid 310, injections of CO₂-saturated brine 312, and injection of storage fluid 316 are depicted in FIG. 3 as arrows at distinct locations on the surface, it is contemplated that these operations may be facilitated by a single bore hole or multiple bore holes.

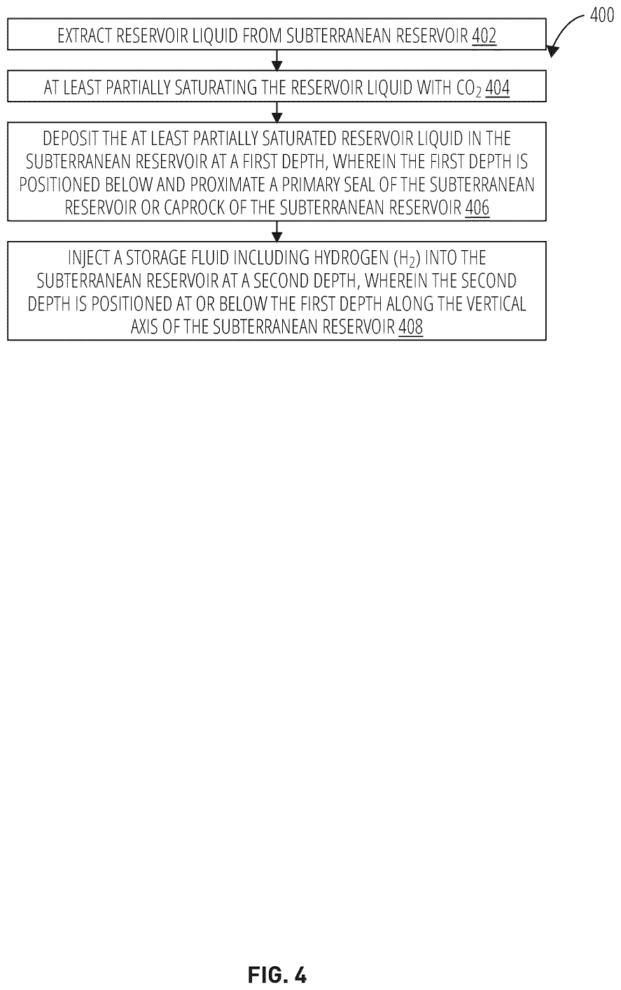

Turning to FIG. 4 and with continued reference to FIG. 3, a method 400 is depicted in accordance with aspects herein. Generally, method 400 includes at least partially saturating a liquid with CO₂, depositing the at least partially saturated liquid in a subterranean reservoir at a first depth, and injecting a storage fluid including Hydrogen (H₂) into the subterranean reservoir at a second depth, wherein the second depth is positioned at or below the first depth along the vertical axis of the subterranean reservoir. Some embodiments of method 400 may be facilitate by the processes and systems of process diagram 300.

Some embodiments of method 400 include, at block 402 extracting reservoir liquid from subterranean reservoir at a first depth along a vertical axis of the subterranean reservoir. For example, a surface facility (e.g., surface facility 304 of FIG. 3) may extract reservoir liquid from the subterranean reservoir using one or more pumps connected to a network of pipes that pass along a bore hole through a seal or caprock (e.g., caprock 302 of FIG. 3) into subterranean reservoir (e.g., subterranean reservoir 306 of FIG. 3). Additionally, or alternatively, some embodiments of method 400 include extracting liquid from a surface water deposit (e.g., lake, river, swamp, sea, or any similar body of water).

In block 404, method 400 includes at least partially saturating the reservoir liquid with CO₂. For example, the brine may be directed to a high-pressure saturator or mixing chamber at the surface facility. The CO₂ gas may be supplied from a high-pressure CO₂ source and can be injected into the brine stream using a gas compressor to ensure that the CO₂ is at a pressure at least equivalent with the subterranean reservoir conditions. The CO₂ may be generated by the surface facility onsite. For example, the CO₂ may be a byproduct of an industrial chemical process, oil refining, energy production, or any other process. In some embodiments, the reservoir liquid is equilibrated with CO₂ at a pressure higher than the hydrostatic pressure of the storage reservoir. For example, the pressure of the storage reservoir may be 5 MPa or higher. In some embodiments, the pressure of the storage reservoir may be 8 MPa or higher. In some embodiments, the pressure of the storage reservoir may be 12 MPa or higher, such as up to 100 MPa. In some embodiments, the reservoir liquid is equilibrated with CO₂ such that the pH is reduced from the reservoir liquid's initial pH to 3-4 pH.

In block 406, method 400 includes depositing the at least partially saturated reservoir liquid in the subterranean reservoir at a predetermined depth. The pressurized CO₂-saturated brine may be transported through a network of high-pressure pipelines from the surface facility to the predetermined depth (e.g., CO₂ brine depth) in the subterranean reservoir. In some embodiments, the predetermined CO₂ brine depth is below and proximate the seal or caprock of the subterranean reservoir. In at least one embodiment, the predetermined depth is at least 1000 m.

In block 408, method 400 includes injecting a storage fluid including H₂ into the subterranean reservoir at another predetermined depth (e.g., H₂ depth). In some embodiments, the storage fluid includes at least 40 vol % H₂. In some embodiments, the predetermined H₂ depth is at least 1000 m. In at least one embodiment, the predetermined H₂ depth is equal to or lower than the CO₂ brine depth.

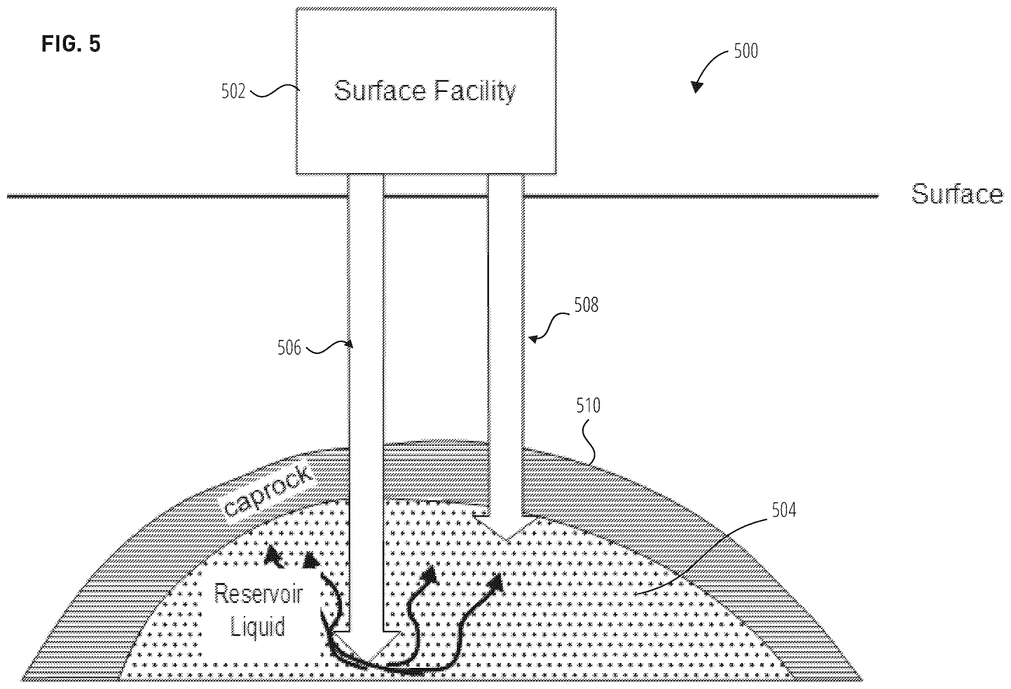

Turning to FIG. 5, a process diagram 500 is depicted in accordance with aspects described herein. In general, process diagram 500 includes the saturation of brine with CO₂. As depicted, CO₂ injection 506 is within the reservoir liquid of the subterranean reservoir. For example, CO₂ gas may be supplied from a high-pressure CO₂ source and can be injected through a network of pipes into the subterranean reservoir. In some embodiments, the pipe system is equipped with high-pressure diffusers that introduce the CO₂ into the subterranean reservoir as fine bubbles. The diffusion of CO₂ with fine bubbles may increase the surface area for dissolution of the CO₂. The CO₂ injection 506 occurs at a CO₂ injection depth. As gaseous CO₂ is buoyant in the reservoir, the CO₂ injection depth in process diagram 500 is proximate or near the bottom of the subterranean reservoir along a vertical access. This facilitates dissolution of the CO₂ throughout the subterranean reservoir while reducing the risk of mobile CO₂ accumulation near the caprock. The saturation of brine with CO₂ lowers the pH of the brine from a native pH to between 3.0 pH and 4.0 pH. The acidification of the brine by CO₂-saturation includes multiple chemical processes that form carbonic acid (H₂CO₃) and dissociation of H₂CO₃ into bicarbonate (HCO₃⁻) and hydrogen ions (H⁺). In some embodiments, the reservoir liquid is equilibrated with CO₂ at a pressure higher than 8 MPa. In some embodiments, the pressure of the storage reservoir may be 12 MPa or higher, such as up to 100 MPa.

Process diagram 500 also includes injecting a storage fluid including H₂ into the subterranean reservoir. The storage fluid includes at least 40 vol % H₂ in some embodiments. In contrast to process diagram 300 of FIG. 3, the H₂ injection depth is above the CO₂ injection depth along a vertical axis of the subterranean reservoir. For example, the H₂ injection depth is below and proximate the seal or caprock.

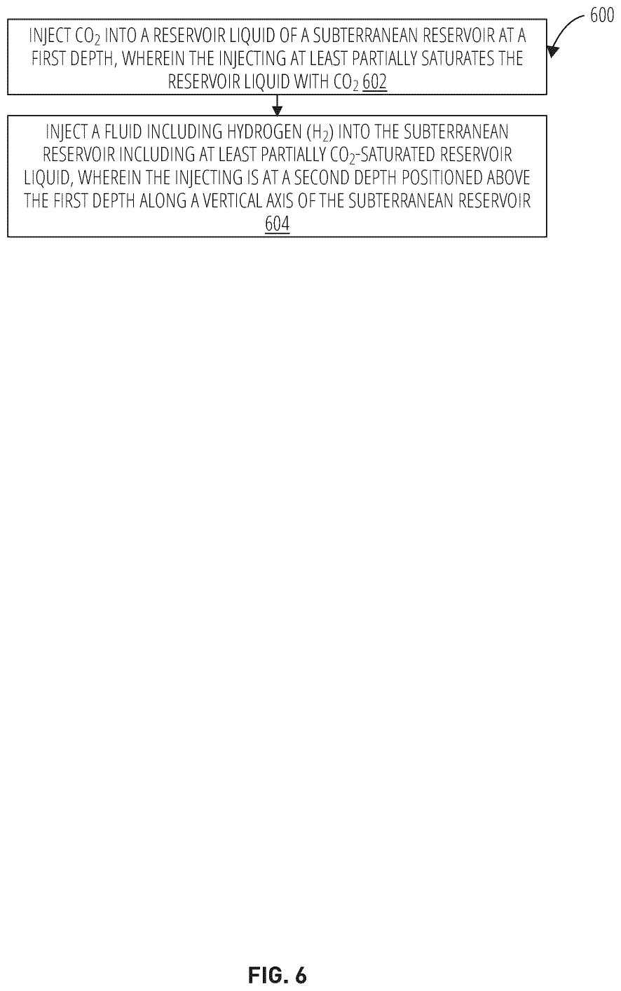

Turning to FIG. 6 and with continued reference to FIG. 5, a method 600 is depicted in accordance with aspects herein. Generally, method 600 includes injecting CO₂ into the reservoir liquid of a subterranean reservoir. CO₂ may be injected until the reservoir liquid is least partially saturated with CO₂. The pH of the CO₂-saturated reservoir liquid is reduced from an initial pH to pH between 3.0 and 4.0 pH. Method 600 also includes injecting a storage fluid including H₂ into the subterranean reservoir at a depth above the CO₂ injection depth along the vertical axis of the subterranean reservoir. Some embodiments of method 600 may be facilitate by the processes and systems of process diagram 500.

In block 602, method 600 includes injecting CO₂ into a reservoir liquid of a subterranean reservoir at a first depth, wherein the injecting at least partially saturates the reservoir liquid with CO₂. The CO₂ gas may be supplied from a high-pressure CO₂ source and can be injected into the brine stream using a gas compressor to ensure that the CO₂ is at a pressure at least equivalent with the subterranean reservoir conditions. The CO₂ may be generated by the surface facility (e.g., surface facility 502) onsite. For example, the CO₂ may be a byproduct of an industrial chemical process, oil refining, energy production, or any other process. In some embodiments, the reservoir liquid is equilibrated with CO₂ at the hydrostatic pressure of the storage reservoir. For example, the pressure of the storage reservoir may be 5 MPa or higher. In some embodiments, the pressure of the storage reservoir may be 8 MPa or higher. In some embodiments, the pressure of the storage reservoir may be 12 MPa or higher, such as up to 100 MPa. In some embodiments, the reservoir liquid is equilibrated with CO₂ such that the pH is reduced from the reservoir liquid's initial pH to 3-4 pH. The CO₂ injection depth may vary based on the subterranean reservoir. In some embodiments, the CO₂ injection depth is proximate or near the bottom of the subterranean reservoir along a vertical access.

In block 604, method 600 injects a storage fluid including H₂ into the subterranean reservoir including at least partially CO₂-saturated reservoir liquid. The injection occurs at an H₂ injection depth positioned above the CO₂ injection depth along a vertical axis of the subterranean reservoir. In some embodiments, the H₂ injection depth is below and proximate at least a portion of cap rock of the subterranean reservoir. In some embodiments, the storage fluid comprises at least 40 vol % H₂.

Certain features have been described using a set of numerical upper limits, a set of numerical lower limits, or a combination of both. It should be appreciated that ranges from any lower limit to any upper limit are contemplated unless otherwise indicated. Certain lower limits, upper limits and ranges appear in one or more claims below. All numerical values take into account experimental error and variations that would be expected by a person having ordinary skill in the art.

The foregoing description of the disclosure illustrates and describes the present methodologies. Additionally, the disclosure shows and describes exemplary methods, but it is to be understood that various other combinations, modifications, and environments may be employed and the present methods are capable of changes or modifications within the scope of the concept as expressed herein, commensurate with the above teachings and/or the skill or knowledge of the relevant art.

While specific elements and steps are discussed in connection to one another, it is understood that any element and/or steps provided herein is contemplated as being combinable with any other elements and/or steps regardless of explicit provision of the same while still being within the scope provided herein. Since many possible embodiments may be made of the disclosure without departing from the scope thereof, it is to be understood that all matter herein set forth or shown in the accompanying drawings is to be interpreted as illustrative and not in a limiting sense.

As used herein and in connection with the claims listed hereinafter, the terminology “any of clauses” or similar variations of said terminology is intended to be interpreted such that features of claims/clauses may be combined in any combination. For example, an exemplary clause 4 may indicate the method/apparatus of any of clauses 1 through 3, which is intended to be interpreted such that features of clause 1 and clause 4 may be combined, elements of clause 2 and clause 4 may be combined, elements of clause 3 and 4 may be combined, elements of clauses 1, 2, and 4 may be combined, elements of clauses 2, 3, and 4 may be combined, elements of clauses 1, 2, 3, and 4 may be combined, and/or other variations. Further, the terminology “any of clauses” or similar variations of said terminology is intended to include “any one of clauses” or other variations of such terminology, as indicated by some of the examples provided above.

Clause 1. A method for fluid storage comprising: extracting reservoir liquid from a subterranean reservoir; at least partially saturating the reservoir liquid with CO₂; depositing at least a portion of the at least partially saturated reservoir liquid in the subterranean reservoir at a first depth, wherein the first depth is positioned below and proximate at least one of a primary seal and a caprock; and injecting a storage fluid including Hydrogen (H₂) into the subterranean reservoir at a second depth, wherein the second depth is positioned at or below the first depth along a vertical axis of the subterranean reservoir.

Clause 2. The method for fluid storage of clause 1, wherein the subterranean reservoir is within a depleted oil field or a depleted gas field.

Clause 3. The method for fluid storage of clause 1 or clause 2, wherein the storage fluid comprises at least 40 vol % H₂.

Clause 4. The method for fluid storage of any of clauses 1 through 3, wherein the reservoir liquid is equilibrated with CO₂ at reservoir pressure which typically ranges between 5 to 100 MPa. or the pH is reduced from an initial pH to 3-4 pH.

Clause 5. The method for fluid storage of any of clauses 1 through 4, wherein the first depth is at least 1000 m.

Clause 6. The method for fluid storage of any of clauses 1 through 5, wherein the second depth is at least 1000 m.

Clause 7. The method for fluid storage of any of clauses 1 through 6, wherein the second depth is at or below the first depth.

Clause 8. The method for fluid storage of any of clauses 1 through 7, wherein at least partially saturating the reservoir liquid with CO₂ comprises pressurizing the CO₂ to reservoir pressure.

Clause 9. The method for fluid storage of any of clauses 1 through 8, wherein the first depth and second depth is proximate at least a portion of caprock of the subterranean reservoir.

Clause 10. The method for fluid storage of any of clauses 1 through 9, wherein at least partially saturating the reservoir liquid with CO₂ comprises reducing pH of the reservoir liquid from an initial pH to a pH in a range of 3-4 pH.

Clause 11. A method for fluid storage comprising: injecting CO₂ into a reservoir liquid of a subterranean reservoir at a reservoir injection depth, wherein the injecting at least reduces the pH of the reservoir liquid from a native pH to a pH in a range of 3-4; and injecting a storage fluid including H₂ into the subterranean reservoir including reservoir liquid at a pH of 3-4, wherein the injecting is at a storage fluid injection depth positioned at or below the reservoir injection depth along a vertical axis of the subterranean reservoir.

Clause 12. The method for fluid storage of clause 11, wherein at least partially saturating the reservoir liquid with CO₂ comprises pressurizing the CO₂ to reservoir pressure.

Clause 13. The method for fluid storage of clause 11 or clause 12, wherein the subterranean reservoir is within a depleted oil field or a depleted gas field.

Clause 14. The method for fluid storage of any of clauses 11 through 13, wherein the storage fluid comprises at least 40 vol % H₂.

Clause 15. The method for fluid storage of any of clauses 11 through 14, wherein the reservoir liquid is equilibrated with CO₂ at a pressure higher than 8 MPa.

Clause 16. The method for fluid storage of any of clauses 11 through 15, wherein the first depth is at least 1000 m.

Clause 17. The method for fluid storage of any of clauses 11 through 16, wherein the second depth is at least 1000 m.

Clause 18. The method for fluid storage of any of clauses 11 through 17, wherein the storage fluid injection depth is proximate at least a portion of cap rock of the subterranean reservoir.

Clause 19. A method for fluid storage comprising: extracting surface liquid from a surface accessible body of water; at least partially saturating surface liquid with CO₂; depositing the at least partially saturated surface liquid in the subterranean reservoir at a first depth, wherein the first depth is positioned below and proximate a primary seal of the subterranean reservoir or caprock of the subterranean reservoir; and injecting a storage fluid including H₂ into the subterranean reservoir at a second depth, wherein the second depth is positioned at or below the first depth along the vertical axis of the subterranean reservoir.

Clause 20. The method for fluid storage of clause 19, wherein the storage fluid comprises at least 40 vol % H₂.

Clause 21. The method for fluid storage of clause 19 or clause 20, wherein at least partially saturating the surface liquid with CO₂ comprises pressurizing the CO₂ to a hydrostatic pressure of the subterranean reservoir.

Clause 22. The method for fluid storage of any if clauses 19 through 21, wherein the hydrostatic pressure of the subterranean reservoir is greater than or equal to 8 MPa.

Clause 23. The method for fluid storage of any if clauses 19 through 22, wherein the subterranean reservoir is within a depleted oil field or a depleted gas field.

Clause 24. The method for fluid storage of any if clauses 19 through 23, wherein the second depth is at least 1000 m.

Clause 25. The method for fluid storage of any if clauses 19 through 24, wherein the second depth is proximate at least a portion of cap rock of the subterranean reservoir.

What is claimed is:

1. A method for fluid storage comprising:
extracting reservoir liquid from a subterranean reservoir;
at least partially saturating the reservoir liquid with CO₂;
depositing at least a portion of the at least partially saturated reservoir liquid in the subterranean reservoir at a first depth, wherein the first depth is positioned below and proximate at least one of a primary seal and a caprock; and
injecting a storage fluid including Hydrogen (H₂) into the subterranean reservoir at a second depth, wherein the second depth is positioned at or below the first depth along a vertical axis of the subterranean reservoir.

2. The method for fluid storage of claim 1, wherein the subterranean reservoir is within a depleted oil field or a depleted gas field.

3. The method for fluid storage of claim 1, wherein the storage fluid comprises at least 40 vol % H₂.

4. The method for fluid storage of claim 1, wherein the reservoir liquid is equilibrated with CO₂ at reservoir pressure which typically ranges between 5 to 100 MPa. or the pH is reduced from an initial pH to 3-4 pH.

5. The method for fluid storage of claim 1, wherein the first depth is at least 1000 m.

6. The method for fluid storage of claim 1, wherein the second depth is at least 1000 m.

7. The method for fluid storage of claim 1, wherein the second depth is at or below the first depth.

8. The method for fluid storage of claim 1, wherein at least partially saturating the reservoir liquid with CO₂ comprises pressurizing the CO₂ to reservoir pressure.

9. The method for fluid storage of claim 1, wherein the first depth and second depth is proximate at least a portion of caprock of the subterranean reservoir.

10. The method for fluid storage of claim 1, wherein at least partially saturating the reservoir liquid with CO₂ comprises reducing pH of the reservoir liquid from an initial pH to a pH in a range of 3-4 pH.

11. A method for fluid storage comprising:
injecting CO₂ into a reservoir liquid of a subterranean reservoir at a reservoir injection depth, wherein the injecting at least reduces the pH of the reservoir liquid from a native pH to a pH in a range of 3-4; and
injecting a storage fluid including Hydrogen (H₂) into the subterranean reservoir including reservoir liquid at a pH of 3-4, wherein the injecting is at a storage fluid injection depth positioned at or below the reservoir injection depth along a vertical axis of the subterranean reservoir.

12. The method for fluid storage of claim 11, wherein at least partially saturating the reservoir liquid with CO₂ comprises pressurizing the CO₂ to reservoir pressure.

13. The method for fluid storage of claim 11, wherein the subterranean reservoir is within a depleted oil field or a depleted gas field.

14. The method for fluid storage of claim 11, wherein the storage fluid comprises at least 40 vol % H₂.

15. The method for fluid storage of claim 11, wherein the reservoir liquid is equilibrated with CO₂ at a pressure higher than 8 MPa.

16. The method for fluid storage of claim 11, wherein the first depth is at least 1000 m.

17. The method for fluid storage of claim 11, wherein the second depth is at least 1000 m.

18. The method for fluid storage of claim 11, wherein the storage fluid injection depth is proximate at least a portion of cap rock of the subterranean reservoir.

19. A method for fluid storage comprising:
extracting surface liquid from a surface accessible body of water;
at least partially saturating surface liquid with CO₂;
depositing the at least partially saturated surface liquid in the subterranean reservoir at a first depth, wherein the first depth is positioned below and proximate a primary seal of the subterranean reservoir or caprock of the subterranean reservoir; and
injecting a storage fluid including Hydrogen (H₂) into the subterranean reservoir at a second depth, wherein the second depth is positioned at or below the first depth along the vertical axis of the subterranean reservoir.

20. The method for fluid storage of claim 19, wherein the storage fluid comprises at least 40 vol % H₂.

21. The method for fluid storage of claim 19, wherein at least partially saturating the surface liquid with CO₂ comprises pressurizing the CO₂ to a hydrostatic pressure of the subterranean reservoir.

22. The method for fluid storage of claim 21, wherein the hydrostatic pressure of the subterranean reservoir is greater than or equal to 8 MPa.

23. The method for fluid storage of claim 19, wherein the subterranean reservoir is within a depleted oil field or a depleted gas field.

24. The method for fluid storage of claim 19, wherein the second depth is at least 1000 m.

25. The method for fluid storage of claim 19, wherein the second depth is proximate at least a portion of cap rock of the subterranean reservoir.
